(19) United States
(12) Patent Application Publication
(10) Pub. No.: US 2019/0184601 A1
(43) Pub. Date: Jun. 20, 2019

(54) SYSTEM AND METHOD FOR LABELING AND MONITORING CEMENTITIOUS COMPOSITES

(71) Applicant: THE BOARD OF REGENTS FOR OKLAHOMA STATE UNIVERSITY, Stillwater, OK (US)

(72) Inventors: Matthew Tyler LEY, Stillwater, OK (US); Kristopher Jacob LEFLORE, Glencoe, OK (US)

(73) Assignee: THE BOARD OF REGENTS FOR OKLAHOMA STATE UNIVERSITY, STILLWATER, OK (US)

(21) Appl. No.: 16/310,570

(22) PCT Filed: Jun. 20, 2017
(86) PCT No.: PCT/US2017/038303

§ 371 Date: Dec. 17, 2018

Related U.S. Application Data
(60) Provisional application No. 62/352,141, filed on Jun. 20, 2016.

(51) Int. Cl. B28B 23/00; B28B 7/00; G01N 33/38

(52) U.S. Cl. CPC B28B 23/0031; G01N 33/383; B28B 7/0094

(57) ABSTRACT

This system includes a concrete identification technology that is used in taking, testing, monitoring, and labeling concrete samples or members using custom labels called a tag. This tag comprises a series of corresponding labels, one for the outside of the form, and on for the inside of the form that becomes part of the concrete sample. These tags are physically bonded to the concrete specimen with only minimal impact on its shape and properties. In some embodiments the tags will contain a hook-and-loop or fiber type surface texture (protrusions) which is placed in the form with the protrusions facing the fresh concrete. The tag can also contain electronic circuitry to gather and track information about the concrete and the surrounding environment. This will eliminate the need to relabel the samples after demolding while also measuring a number of other parameters about the concrete and its performance.

### CROSS REFERENCE TO RELATED APPLICATIONS

This application claims the benefit of U.S. Provisional Patent Application Ser. No. 62/352,141 filed on Jun. 20, 2016, and incorporates said provisional application by reference into this document as if fully set out at this point.

### TECHNICAL FIELD

This disclosure relates generally to the taking, testing, monitoring, and labeling concrete specimens for quality control testing or concrete members.

### BACKGROUND

Millions of concrete specimens are taken annually and the labeling, and relabeling upon demolding, has always been a challenge of the concrete construction industry. The results of these tests determine payment for supplied concrete so these test results are important.

Furthermore, many members are precast or premanufactured out of concrete and then shipped to the job site for usage. Identification of these members and determining the details of their construction is an important and challenging task.

It also may be helpful to label members that are cast-in-place on a job site to establish payment, communicate important details such as a defect, superior property, location within a BIM model or perhaps as a guide so that others know where they need to complete their next set of work.

Sample and member labeling and monitoring has always caused problems in the concrete industry for a few different reasons. For example, the conventional approach is to demold the concrete specimen from the form it was cast in and relabel it using a marker, tape, or grease pen. This is done manually. If the writing cannot be read, if it becomes worn away, or if the sample or member is never labeled then this can cause significant problems. Furthermore, sometimes there is only a limited amount of space where a label could be added to the member and it can be challenging to modify these labels to fit and still communicate effectively.

It is also possible to fix a label or radio-frequency identification (hereinafter “RFID”) tag to the surface of concrete after it has been removed from the form. However, often these samples and members are placed in extreme environments (such as saturated lime water, deicing salts, freezing environments, and severe drying, etc.) and this can cause the connection to fail and then the sample or member is no longer labeled. A solution is needed that can label the sample or member as it is being cast and provide a way to fix the label securely to bond the label to the surface that is robust and which does not significantly change the sample or member dimensions.

Before proceeding to a description of the present invention, however, it should be noted and remembered that the description of the invention which follows, together with the accompanying drawings, should not be construed as limiting the invention to the examples (or embodiments) shown and described. This is so because those skilled in the art to which the invention pertains will be able to devise other forms of this invention within the ambit of the appended claims.

### SUMMARY OF THE INVENTION

According to an embodiment, there is provided a system of labeling or sensing for concrete samples or members that does not suffer from the problems of previous approaches.

This system which includes an internal “tag” solves many of the issues commonly experienced with labeling concrete specimens and elements.

One embodiment is a concrete labeling technology that is used to track concrete samples or elements using custom labels. An embodiment uses a series of corresponding labels, one for the outside of the form, and one for the inside of the form that becomes part of the concrete sample. It is also possible to just use the tag inside of the forms and without the corresponding external label. The corresponding inside tags are physically bonded to the concrete specimen in a way that creates only a minimal impact on the shape and properties of the concrete. This embodiment will eliminate the need to relabel the samples after demolding. It will also allow many more properties of the concrete to be added to the label such as the conditions, locations, details of creation, materials, and performance to be effectively logged over time.

The foregoing has outlined in broad terms some of the more important features of the invention disclosed herein so that the detailed description that follows may be more clearly understood, and so that the contribution of the instant inventors to the art may be better appreciated. The instant invention is not to be limited in its application to the details of the construction and to the arrangements of the components set forth in the following description or illustrated in the drawings. Rather, the invention is capable of other embodiments and of being practiced and carried out in various other ways not specifically enumerated herein. Finally, it should be understood that the phraseology and terminology employed herein are for the purpose of description and should not be regarded as limiting, unless the specification specifically so limits the invention.

### BRIEF DESCRIPTION OF THE DRAWINGS

These and further aspects of the invention are described in detail in the following examples and accompanying drawings.

FIG. 1 contains an exterior view of a concrete form suitable for use with an embodiment.

FIG. 2 contains an interior view of a form with an embodiment of the inventive tag installed.

FIG. 3 contains a view of an upper side of an embodiment of the instant device.

FIG. 4 contains a view of a lower side of an embodiment of the instant device.

FIG. 5 contains a view of the embodiment bonded to the surface of a concrete specimen.

FIG. 6 contains a schematic view of an embodiment that contains a circuit board.

### DETAILED DESCRIPTION

While this invention is susceptible of embodiment in many different forms, there is shown in the drawings, and will herein be described hereinafter in detail, some specific embodiments of the instant invention. It should be understood, however, that the present disclosure is to be considered an exemplification of the principles of the invention and is not intended to limit the invention to the specific embodiments or algorithms so described.

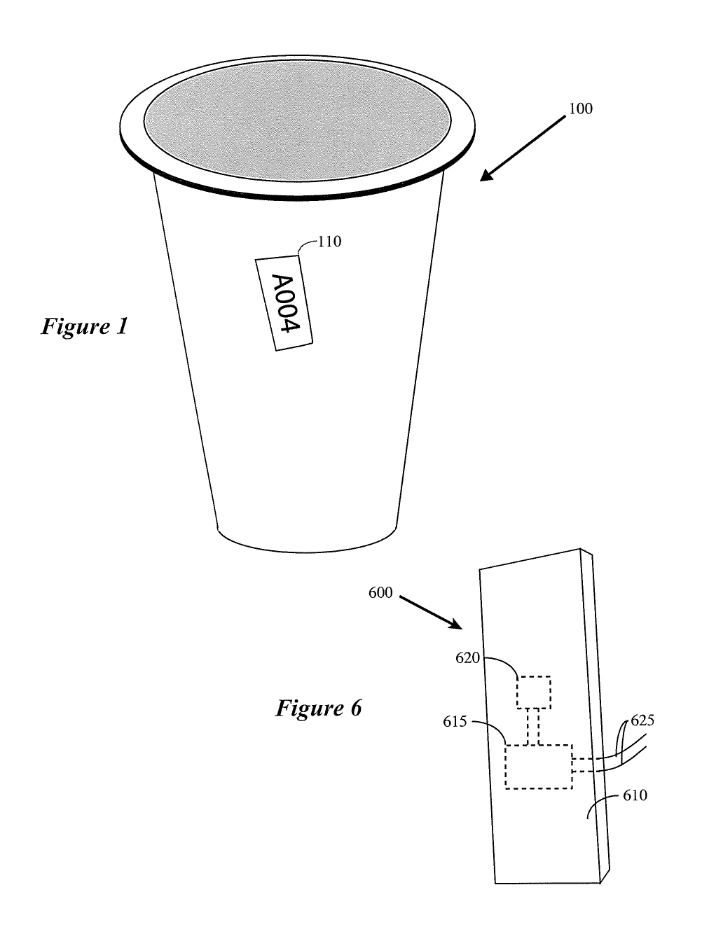

In one embodiment and as is illustrated in FIG. 1, an adhesive exterior label 110 is affixed to a form 100 that is designed to receive semi-liquid concrete samples as is conventionally done, the purpose of the exterior label 110 being to uniquely identifying the contents of the form 100 through the use of a series of characters or other identifiers. This identifier is conventionally placed on the outside of the form 100 to identify the material while the concrete sample hardens within the form 100. The exterior label 110 could also include a bar code, RFID tag, QR code, etc. Of course, adhesive is just one way of affixing the label 110 to the form. Those of ordinary skill in the art will understand that that there are numerous ways of attaching the label to a form that don't involve adhesive, with the particular type of attachment potentially depending on the type of form that is used (e.g., nails, staples, screws, magnets, hook-and-loop, etc.). In some embodiments the label 110 will be removably attached. In other instances it could be permanently attached. As such, when it is said herein that the label 110 is attached or attachable to a surface, that language should be broadly interpreted to include any conventional or unconventional means suitable for attaching that label to the chosen form.

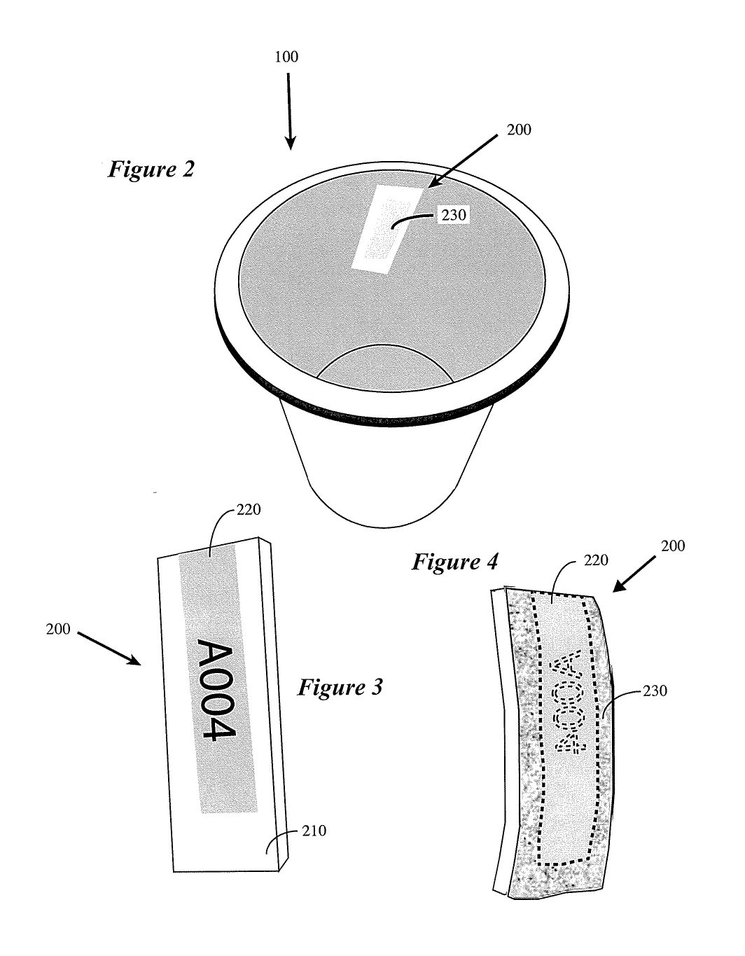

An embodiment of the corresponding inventive interior tag 200 (FIGS. 2 and 3) has an adhesive (in some cases a contact adhesive) applied to one face (the upper face 210 of the tag 200 for purposes of specificity hereinafter) that is designed to allow it to be removably affixed to an interior wall or bottom of the form 100. The strength of the adhesive must be chosen such that the tag 200 remains in place while fresh concrete is added but will release when the form is removed.

According to the embodiment of FIG. 3, the face 210 additionally contains a descriptive label 220 that covers at least a portion of its surface and which contains information that might be a printed, embossed, etc. on the label 220. The descriptive label 220 and/or the tag 200 to which it is attached could also include a bar code, RFID tag, QR code or a microcontroller and sensor(s) to measure the immediate or long-term performance of the concrete over time. If the chosen adhesive is relatively clear (e.g., transparent or translucent) the label 220 might be permanently adhered to the upper face 210 of the tag and adhesive applied on top of it.

In other embodiments, a piece of double sided tape might be used on the surface with the written information between the tag 200 and the form 100. In some embodiments, if the tape is not transparent it could be removably attached to the tag 200 so that after the concrete is removed from the form the label 220 could be made visible again. Of course, if the label is, for example, an RFID chip, removal of the tape would not be necessary. In either case, if tape with two adhesive sides is used to secure the tag 200 to the form, the tape will considered to be a part of the tag 200 unless specifically indicated otherwise. Again, the strength of the adhesive on the tape must be chosen such that the tag 200 remains in place while fresh concrete is added but will release when the form is removed, thereby revealing the label 220 that has become bonded to the concrete.

Note that for purposes of the instant disclosure, when the term “adhesive” is used herein that term should be broadly construed to be contact adhesive (clear, translucent, or opaque), or other adhesive or a mechanical mechanism (e.g., a hook and loop system) that secures the tag 200 to the side of the form 100 while the fresh concrete is being added to the form and then allows the tag 200 to be released from the form after it has bonded to the concrete sample.

Preferably the label 220 will be permanently secured to the upper face 210 of the tag 200. In some embodiments the code printed thereon will match the label 110 on the exterior of the form 100. Of course, that is not strictly necessary but will be useful in many embodiments. A central role of the descriptive label 220 is to allow the concrete specimen or member to be identified after it is removed from the form 100 as described below. The tag's upper surface 210 will preferably be generally planar and pliable so as allow it to mate securely with the interior of the form 100.

The lower face 230 of the tag 200 embodiment of FIG. 4 comprises a material that is able to reliably bond to the fresh concrete when it is poured into the form 100. In some embodiments, the textured surface of the lower face 230 will take the form of a material such as the fibers or loops side of a hook-and-loop/Velcro®-type product or any other textured surface. This surface 230 might have fibers, loops, hooks, fibers of various lengths, or, more generally, protrusions on the surface that extend toward the interior of the form 100. For purposes of the instant disclosure, the word “protrusions” will be used in a general sense to describe any sort of surface texture that is found on at least a portion of the surface 230, that extends toward the interior of the form after the tag 200 is affixed to it, and that can become embedded in the outer surface of fresh concrete and become permanently bonded thereto after the concrete hardens.

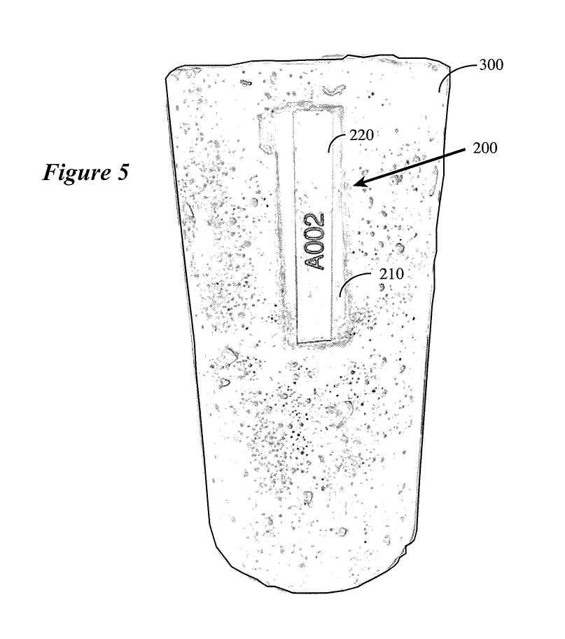

Regardless of the procedure used, what is important is that the texture of the surface 230 be such that the tag 200 becomes reliably embedded, bonded, or otherwise attached to the concrete 300 after it has hardened as shown in the example of FIG. 5.

Once the form 100 is removed, the textured material of the tag face 230 provides a secure bond to the concrete that is stronger than the adhesive on the surface of the opposite face 210. This means the tag 200 will separate from the form 100 and remain attached to the surface of the concrete. The descriptive label 220 will now be visible and will continue to serve as an identifier of that particular concrete member or sample after it has been extracted from the form 100. The inventive tag 200 has a minimal surface profile and so will not impact the shape, volume, or mechanical properties of the extracted specimen while still providing a means of identifying the sample.

By way of example, the tag 200 can have numbers, bar codes, QR codes, etc., printed on the label 220 to help identify the sample. Barcodes and the like could be easily scanned to help track the samples. The tag 200 may also contain a passive or active RFID, microcontroller or other electronic component that can digitally record this information. An application for a mobile device (e.g., a smart phone or tablet) could be developed that allows all of the construction details or any other helpful information to be recorded for the concrete specimen or member and tie this to the information printed on the label or to the unique identifier communicated by the RFID, microcontroller or other electronic component contained within the tag 200. This information could be placed in long-term storage and would be of great value to the user.

Additionally and according to other embodiments, the tag could contain passive or active RFID, microcontrollers, or other electronic components that can use sensor technology to measure a number of properties/parameters of the concrete or its environment while it is being placed or while those parameters change over time. These may include: conductivity, resistivity, impedance, temperature, moisture content, cracking, ion concentration, pH, acceleration from shock, and overall effectiveness of the concrete. The tag 200 would provide a method to obtain these measurements from the concrete while also labeling the material. Note that, for purposes of the instant disclosure, when it is said that parameters of the concrete are measured, that phrase should be broadly interpreted to include instances where the measured parameter includes a measurement of the environment surrounding or proximate to where the concrete is located.

Further, in some embodiments (e.g., FIG. 6), a tag 600 might include a circuit board 615 and power supply/battery 620 to allow additional/real-time information to be obtained about the curing concrete (e.g., wirelessly via Bluetooth®). In some embodiments, sensors that report temperature (e.g., using a thermocouple or other device or label that indicates an approximate temperature or that reacts to a temperature change and changes color such as a liquid crystal thermometer), resistivity/conductivity/impedance (e.g., using probes of a known spacing and measuring the response to controlled electrical signals over time), moisture content/humidity (e.g., by comparing change in electrical signal of known materials with moisture content), pH/other ion measurement (e.g. by comparing change in electrical signal of specially designed sensing links), cracking or strain (e.g. by measuring the change in electrical signal of a material with deformation), and other parameters of the concrete might be made a part of the tag 600. In some embodiments, the sensor(s) might be readable by a microprocessor in the circuit board 615. In still other embodiments, wire leads 625 might be extended from the tag 600 for subsequent use (e.g., when reading any of the provided sensors), in which case the circuit board 615 might be optional if the sensors are in electrical communication with the wire leads 625. Sensors such as an impact sensor (e.g., accelerometer) might also be made a part of the tag 600 and used in subsequent testing of the sample or an indication of the handling of the sample or member. In this sort of embodiment, it will be important (as before) that the face 610 of the tag 600 have an adhesive that will allow it to be removably attached to the form and maintained in place while the fresh concrete is being added and then detached from the form after the opposite side of the tag has bonded with the hardened concrete in the form.

Still further, it should be noted that although one application is concrete samples that are used to test the properties of the hardened material, it should be clear that this approach could also be used with pre-formed or precast items or cast-in-place, columns, slabs, etc.

It is to be understood that the terms “including”, “comprising”, “consisting” and grammatical variants thereof do not preclude the addition of one or more components, features, steps, or integers or groups thereof and that the terms are to be construed as specifying components, features, steps or integers.

If the specification or claims refer to “an additional” element, that does not preclude there being more than one of the additional element.

It is to be understood that where the claims or specification refer to “a” or “an” element, such reference is not be construed that there is only one of that element.

It is to be understood that where the specification states that a component, feature, structure, or characteristic “may”, “might”, “can” or “could” be included, that particular component, feature, structure, or characteristic is not required to be included.

Where applicable, although state diagrams, flow diagrams or both may be used to describe embodiments, the invention is not limited to those diagrams or to the corresponding descriptions. For example, flow need not move through each illustrated box or state, or in exactly the same order as illustrated and described.

Methods of the present invention may be implemented by performing or completing manually, automatically, or a combination thereof, selected steps or tasks.

The term “method” may refer to manners, means, techniques and procedures for accomplishing a given task including, but not limited to, those manners, means, techniques and procedures either known to, or readily developed from known manners, means, techniques and procedures by practitioners of the art to which the invention belongs.

For purposes of the instant disclosure, the term “at least” followed by a number is used herein to denote the start of a range beginning with that number (which may be a range having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. Terms of approximation (e.g., “about”, “substantially”, “approximately”, etc.) should be interpreted according to their ordinary and customary meanings as used in the associated art unless indicated otherwise. Absent a specific definition and absent ordinary and customary usage in the associated art, such terms should be interpreted to be ±10% of the base value.

When, in this document, a range is given as “(a first number) to (a second number)” or “(a first number)-(a second number)”, this means a range whose lower limit is the first number and whose upper limit is the second number. For example, 25 to 100 should be interpreted to mean a range whose lower limit is 25 and whose upper limit is 100. Additionally, it should be noted that where a range is given, every possible subrange or interval within that range is also specifically intended unless the context indicates to the contrary. For example, if the specification indicates a range of 25 to 100 such range is also intended to include subranges such as 26-100, 27-100, etc., 25-99, 25-98, etc., as well as any other possible combination of lower and upper values within the stated range, e.g., 33-47, 60-97, 41-45, 28-96, etc. Note that integer range values have been used in this paragraph for purposes of illustration only and decimal and fractional values (e.g., 46.7-91.3) should also be understood to be intended as possible subrange endpoints unless specifically excluded.

It should be noted that where reference is made herein to a method comprising two or more defined steps, the defined steps can be carried out in any order or simultaneously (except where context excludes that possibility), and the method can also include one or more other steps which are carried out before any of the defined steps, between two of the defined steps, or after all of the defined steps (except where context excludes that possibility).

Further, it should be noted that terms of approximation (e.g., “about”, “substantially”, “approximately”, etc.) are to be interpreted according to their ordinary and customary meanings as used in the associated art unless indicated otherwise herein. Absent a specific definition within this disclosure, and absent ordinary and customary usage in the associated art, such terms should be interpreted to be plus or minus 10% of the base value.

Still further, additional aspects of the instant invention may be found in one or more appendices attached hereto and/or filed herewith, the disclosures of which are incorporated herein by reference as if fully set out at this point.

Thus, the present invention is well adapted to carry out the objects and attain the ends and advantages mentioned above as well as those inherent therein. While the inventive device has been described and illustrated herein by reference to certain preferred embodiments in relation to the drawings attached thereto, various changes and further modifications, apart from those shown or suggested herein, may be made therein by those of ordinary skill in the art, without departing from the spirit of the inventive concept the scope of which is to be determined by the following claims.

What is claimed is:

1. A tag for use on a concrete member or sample hardened within a concrete form, said tag having a first side and second side,
wherein said first side of said tag has a plurality of protrusions suitable for embedding in a surface of fresh concrete placed in a form such that said tag becomes bonded to said fresh concrete after it hardens into the concrete member or sample, and
wherein said second side of said tag has an adhesive thereon that removably attaches said tag to the form and detaches when the concrete member or sample is demolded, and
said second side of said tag further comprising a descriptive label thereon that becomes visible after the concrete member or sample has been demolded.

2. The tag for use on a concrete member or sample according to claim 1 further comprising an RFID chip incorporated into said tag.

3. The tag for use on a concrete member or sample according to claim 1 further comprising a bar code affixed to an outside of the concrete form containing the concrete member or sample.

4. The tag for use on a concrete member or sample according to claim 1 further comprising at least one sensor for sensing a parameter of said concrete member or sample.

5. The tag for use on a concrete member or sample according to claim 1 further comprising at least one sensor for sensing an environment surrounding said concrete member or sample.

6. The tag for use on a concrete member or sample according to claim 1, wherein said protrusions comprise at least one of fibers, loops, and hooks.

7. A tag for use on a concrete member or sample hardened within a concrete form, said tag having a first side and second side, wherein
said first side of said tag has a plurality of protrusions suitable for embedding in a surface of fresh concrete in the form such that said tag becomes bonded to said fresh concrete after it hardens into the concrete member or sample, and
said second side of said tag said has an adhesive thereon that removably attaches said tag to the form and detaches from the form when the concrete member or sample is demolded, and
a sensor within said tag for sensing one or more parameters of the concrete member or sample or for sensing an environment around the member or sample.

8. The tag for use on a concrete member or sample according to claim 7 further comprising an RFID chip incorporated into said tag.

9. The tag for use on a concrete member or sample according to claim 7 wherein said sensor is one or more of a conductivity sensor, a resistivity sensor, an impedance sensor, a temperature sensor, a moisture content sensor, a cracking sensor, an ion concentration sensor, a pH sensor, accelerometer, and a sensor that measures an overall effectiveness of the concrete member or sample.

10. The tag for use on a concrete member or sample according to claim 7 wherein said sensor is one or more of a temperature sensor, a moisture sensor, a humidity sensor, an ion concentration sensor, a radiant heat sensor, and a wind speed sensor.

11. A method of labeling a concrete member or sample, comprising the steps of:
a. obtaining a tag having a first side and a second side, said first side comprising a plurality of protrusions suitable for embedding in fresh concrete, and said second side having an identifying label thereon;
b. removably affixing said second side of said tag to an inside of a concrete form;
c. filling said concrete form with a quantity of the fresh concrete;
d. allowing said quantity of fresh concrete to harden within said concrete form, thereby forming said concrete member or sample and bonding said tag to said hardened concrete; and
e. demolding said concrete member or sample and said tag from said form.

12. The method according to claim 11, wherein said protrusions are selected from the group consisting of fibers, loops, and hooks.

13. The method according to claim 11, wherein said second side of said tag is adhered to the inside of the concrete form.

14. The tag for use on a concrete member or sample according to claim 11 further comprising a corresponding bar code affixed to an outside of the concrete form containing the concrete member or sample.

15. The method according to claim 11, wherein said tag further comprises an RFID chip incorporated into said tag.

16. The method according to claim 11, wherein said tag further comprises at least one sensor for sensing a parameter of said concrete member or sample.

17. The method according to claim 16, wherein said sensor comprises a sensor for sensing an environment surrounding said concrete member or sample.
